(19) United States
(12) Patent Application Publication
(10) Pub. No.: US 2005/0100938 A1
(43) Pub. Date: May 12, 2005

(54) Vertical impedance sensor arrangement and method for producing a vertical impedance sensor arrangement

(75) Inventors: Franz Hofmann, Munchen (DE); Richard Johannes Luyken, Munchen (DE); Wolfgang Roesner, Ottobrunn (DE)

Correspondence Address: DARBY & DARBY P.C., P. O. BOX 5257, NEW YORK, NY, 10150-5257, US

(73) Assignee: Infineon Technologies AG, Munich (DE)

(21) Appl. No.: 10/939,319

(22) Filed: Sep. 9, 2004

Related U.S. Application Data
(63) Continuation of application No. PCT/DE03/00828, filed on Mar. 14, 2003.

(30) Foreign Application Priority Data
Mar. 14, 2002 (DE) 102 11 358.0

(51) Int. Cl. C12Q 1/68

(52) U.S. Cl. 435/6; 435/287.2; 422/82.02

(57) ABSTRACT

Vertical impedance sensor arrangement including a substrate, a first electrically conductive structure having a first uncovered surface and being arranged in and/or on the substrate, a spacer arranged above the substrate and/or at least partially on the first electrically conductive structure, a second electrically conductive structure having a second uncovered surface and being arranged on the spacer, and capture molecules, which are immobilized on the first and on the second uncovered surface, are set up such that particles to be detected hybridize with the capture molecules. The spacer is formed separately from the substrate, and the thickness of the spacer is defined by means of a deposition method.

### CROSS-REFERENCE TO RELATED APPLICATION

This application is a continuation of International Patent Application Serial No. PCT/DE03/00828, filed Mar. 14, 2003, which published in German on Sep. 25, 2003 as WO 03/078991, and is incorporated herein by reference in its entirety.

### FIELD OF THE INVENTION

The invention relates to a vertical impedance sensor arrangement and a method for producing a vertical impedance sensor arrangement.

### BACKGROUND OF THE INVENTION

The detection of molecular biopolymers using a biochip arrangement is of great interest in many areas of chemical, biological and pharmaceutical analysis.

The prior art discloses providing molecules to be detected with a fluorescent label. After the particles to be detected have hybridized with capture molecules immobilized on a sensor surface, electromagnetic primary radiation can be radiated onto the hybridized particles. The hybridization event can be detected by detecting a fluorescent radiation that is remitted by the fluorescent labels after absorption of the primary radiation.

However, spectroscopic methods in which the intensity of a fluorescent radiation or an electromagnetic radiation intensity that is attenuated on account of an absorption of radiation is detected are complicated and often difficult in terms of preparation. Furthermore, marking capture molecules with fluorescent labels is susceptible to errors. Devices for generating or for detecting electromagnetic radiation are expensive and complicated.

Providing capture molecules or particles to be detected with a fluorescent label is dispensed with when using an electrical detection method for detecting hybridization events. Electrical methods for detecting biomolecules are described for example in the following documents:
Hintsche, R., Paeschke, M., Uhlig, A., Seitz, R. (1997) “Microbiosensors using Electrodes made in Si-technology”, Frontiers in Biosensorics, Fundamental Aspects, Scheller, F. W., Schubert, F., Fedrowitz, J. (eds.), Birkhauser Verlag Basle, Switzerland, pp. 267 283;
Van Gerwen, P. (1997) “Nanoscaled Interdigitated Electrode Arrays for Biochemical Sensors”, IEEE, International Conference on Solid-State Sensors and Actuators, Jun. 16-19, 1997, Chicago, pp. 907-910;
Paeschke, M., Dietrich, F., Uhlig, A., Hintsche, R. (1996) “Voltammetric Multichannel Measurements Using Silicon Fabricated Microelectrode Arrays”, Electroanalysis, Vol. 7, No. 1, pp. 1-8;
WO 97/21094; and
DE 19610115 A1.

FIGS. 1A and 1B show a sensor known from the prior art, in the case of which a hybridization event is detected electrically.

The sensor 100 has two electrodes 101, 102 made of gold material, which are embedded in an insulator layer 103 made of electrically insulating material. Connected to the electrodes 101, 102 are electrode terminals 104, 105, by means of which an electrical potential can be applied to the electrodes 101, 102. The electrodes 101, 102 are planar electrodes. DNA probe molecules 106 are immobilized on each electrode 101, 102.

If an electrolyte 107 contains DNA strands 108 with a base sequence which is complementary to the sequence of the DNA probe molecules 106, that is to say which sterically match the probe or capture molecules 106 in accordance with the key/lock principle, then these DNA strands 108 hybridize with the DNA probe molecules 106, as shown in FIG. 1B.

Hybridization of a DNA probe molecule 106 and a DNA strand 108 takes place only when the sequences of the respective DNA probe molecule 106 and of the corresponding DNA half strand 108 are complementary to one another.

If hybridization takes place, then the value of the impedance between the electrodes 101, 102 changes. This changed impedance is detected by applying a suitable electrical signal to the electrode terminals 104, 105 and by detecting the associated electric current.

A description is given below, with reference to FIG. 2A to FIG. 2C of a method known from the prior art for detecting macromolecular biomolecules using a reduction/oxidation recycling process, also referred to hereinafter as redox recycling process.

FIG. 2A shows a biosensor 200 having a first electrode 201 and a second electrode 202, which are applied to an insulator layer 203. A holding region 204 made of gold material is applied on the first electrode 201. The holding region 204 serves for immobilizing DNA probe molecules 205 on the first electrode 201. Such a holding region is not provided on the second electrode 202.

If DNA strands 207 having a sequence which is complementary to the sequence of the immobilized DNA probe molecules 205 are intended to be detected by means of the biosensor 200, then the biosensor 200 is brought into contact with a solution to be examined, for example an electrolyte 206, in such a way that DNA strands 207 possibly contained in the solution 206 to be examined can hybridize with a complementary sequence to the sequence of the DNA probe molecules 405.

FIG. 2B shows a scenario in accordance with which DNA strands 207 to be detected are contained in the solution 206 to be examined, one of which DNA strands has hybridized with a DNA probe molecule 205. The DNA strands 207 in the solution to be examined are marked with an enzyme 208, with which it is possible to cleave molecules described below into electrically charged partial molecules. It is customary to provide a considerably larger number of DNA probe molecules 205 than there are DNA strands 207 to be determined contained in the solution 206 to be examined.

After the DNA strands 207 possibly contained in the solution 206 to be examined, together with the enzyme 208, are hybridized with the immobilized DNA probe molecules 205, the biosensor 200 is rinsed, as a result of which those DNA strands at which a hybridization event has not taken place are removed and the biosensor 200 is cleaned of the solution 206 to be examined. A rinsing solution used for rinsing has an electrically uncharged substance added to it, which contains molecules that can be cleaved by means of the enzyme 208, into a first partial molecule 210 having a negative electrical charge and into a second partial molecule having a positive electrical charge.

As shown in FIG. 2C, the negatively charged first partial molecules 210 are attracted to the positively charged first electrode 201, which is indicated by means of an arrow 211 in FIG. 2C. The negatively charged first partial molecules 210 are oxidized at the first electrode 201, which has a positive electrical potential, and are attracted as oxidized partial molecules 213 to the negatively charged second electrode 202, where they are reduced again. The reduced partial molecules 214 again migrate to the positively charged electrode 201. In this way, an electric circulating current is generated, which is proportional to the number of charge carriers respectively generated by means of the enzymes 208.

The known impedance methods have the disadvantage that an electrical signal that is only very small in each case can be evaluated. The change in the electric field on account of DNA half strands hybridizing with capture molecules immobilized on a sensor surface is very small.

As known from Van Gerwen, P., Laureyn, W., Laureys, W., Huyberechts, G., De Beeck, M. O., Baert, K., Suls, J., Sansen, W., Jacobs, P., Hermans, L., Mertens, R. (1998) “Nanoscaled interdigitated electrode arrays for biochemical sensors,” Sensors and actuators B 49:73 80, it is possible to improve the sensitivity of a sensor arrangement by reducing the lateral dimension of a planar arrangement of sensor electrodes.

FIG. 3A shows a sensor arrangement 300, in which a first electrode 302 is arranged at a distance of 1 μm away from a second electrode 303 on a substrate 301. Capture molecules 304 are immobilized on the electrodes 302, 303 and have hybridized with particles 305 to be detected, in accordance with the operating state shown in FIG. 3A. Furthermore, FIG. 3A shows first to fourth electric field curves 306a to 306d that are obtained from simulation calculations and specify the strength of the electric field between the electrodes 302, 303 at what distance from the surface of the substrate 301. In the case of a lateral extent of 1 μm between the two electrodes 302, 303, only a very small proportion of the electric field lies in a surrounding region of the electrodes 302, 303 near the surface, which region is critically influenced by hybridization events. Therefore, the sensor arrangement 300 shown in FIG. 3A has a detection sensitivity requiring improvement.

FIG. 3B shows a sensor arrangement 310, which essentially has the same components as the sensor arrangement 300 shown in FIG. 3A. However, the lateral distance between adjacent electrodes 311 and 312, 312 and 313, 313 and 314 is 0.2 μm in each case. Once again, first to fourth electric field curves 315a to 315d are depicted in FIG. 3B. On account of the reduced dimension of the electrodes 311 to 314 compared with the sensor arrangement 300, a considerably larger proportion of the field therefore lies in a region near the surface between the electrodes 311 and 312 than in the scenario of FIG. 3A.

Clearly, a change in the electric field is detected during an impedance measurement. A reduced dimension of electrodes of an impedance sensor arrangement brings about an increase in the detection sensitivity.

The sensor arrangements 300, 310 shown in FIG. 3A, FIG. 3B are produced using a method appertaining to technology. In particular, the electrodes are produced using lithography methods and etching methods. However, it is technologically difficult to produce spacings of less than 200 nm using a lithography method. This requires a very expensive, demanding lithography. On account of fundamental physical restrictions such as undesirable diffraction phenomena in the case of optical lithography with a mask having a very small dimension or on account of the relatively high inaccuracy in the case of a lithographic patterning (errors of 20 nm or worse), it is extremely difficult to form a sensor arrangement with electrodes having a sufficiently small dimension which are produced using a patterning method.

Niwa, O., Morita, M., Tabei, H. (1991) “Highly Sensitive and Selective Voltammetric Detection of Dopamine with Vertically Separated Interdigitated Array Electrodes” Electroanalysis 3:163 168, and Rehacek, V., Novotny, I., Ivanic, R., Breternitz, V., Spiess, L., Knedlik, C. H., Tvarocek, V. (2000) “Vertically Arranged Microelectrode Array for Electrochemical Sensing” Third International EuroConference on Advanced Semiconductor Device and Microsystems, Smolenice Castle, Slovakia, Oct. 16-18, 2000, disclose interdigital arrangements of vertically arranged electrodes which are set up for the detection of redox-active particles. In this case, by applying an electrical voltage to the electrodes, a redox-active species is attracted to the electrodes on account of an electrical force and detected in the form of an electric current.

DE 100 15 547 A1 discloses methods for detecting molecules by means of impedance spectroscopy and an apparatus for carrying out these methods.

WO 01/75151 A2 discloses a method for detecting macromolecular biopolymers by means of an electrode arrangement.

WO 97/21094 A1 discloses a sensor for identifying molecular structures within a sample.

WO 88/09499 A1 discloses an optimized capacitance sensor for chemical analysis and measurement.

WO 01/43870 A2 discloses a column- and row-addressable high-density biochip arrangement.

### SUMMARY OF THE INVENTION

The invention is based on the problem of providing a sensor arrangement for detecting particles by means of the particles hybridizing with immobilized capture molecules, which sensor arrangement has an improved detection sensitivity.

The problem is solved by means of a vertical impedance sensor arrangement and by means of a method for producing a vertical impedance sensor arrangement.

The vertical impedance sensor arrangement according to the invention has a substrate, a first electrically conductive structure having a first uncovered surface, which first electrically conductive structure is arranged in and/or on the substrate, and a spacer arranged above the substrate and/or at least partially on the first electrically conductive structure. A second electrically conductive structure having a second uncovered surface is arranged on the spacer. Furthermore, the vertical impedance sensor arrangement has capture molecules that are immobilized on the first and second uncovered surfaces and are set up in such a way that particles to be detected can hybridize with said capture molecules.

In accordance with the method according to the invention for producing a vertical impedance sensor arrangement, a first electrically conductive structure having a first uncovered surface is formed in and/or on a substrate. Furthermore, a spacer is formed above the substrate and/or at least partially on the first electrically conductive structure. Moreover, a second electrically conductive structure having a second uncovered surface is formed on the spacer. Capture molecules are immobilized on the first uncovered surface and on the second uncovered surface, said capture molecules being set up in such a way that particles to be detected can hybridize with said capture molecules.

In the case of the vertical impedance sensor arrangement according to the invention, the distance between the sensor electrodes, that is to say the first and second electrically conductive structures, is defined by means of a vertical arrangement. By depositing a layer as a spacer, it is possible to set the distance between the electrodes with a very high accuracy. A fundamental idea of the invention is to be seen in the fact that a thickness of the spacer is prescribed by means of a deposition method, and not using a patterning method as in the prior art. Appropriate deposition methods are, in particular, an atomic layer deposition method or a chemical vapor phase epitaxy method. Particularly in the case of the atomic layer deposition method (ALD method), it is possible to set the accuracy of a deposited layer down to an accuracy of as much as one atomic layer, that is to say down to an accuracy of a few angstroms. Therefore, it is possible to set a distance between the sensor electrodes of a sensor arrangement with a very high accuracy. A minimum distance between the two sensor electrodes of less than 100 nm can therefore be achieved without any problems.

Using the effect explained with reference to FIG. 3A, FIG. 3B, in the case of a reduced distance between the sensor electrodes, the electric field distribution between the sensor electrodes is influenced to a particularly great extent by a hybridization event. As a result, the detection sensitivity of the vertical impedance sensor arrangement according to the invention is significantly increased compared with the prior art. Moreover, the vertical impedance sensor arrangement according to the invention can be produced by means of a simple lithography and a simple lift-off method. Therefore, the production of the vertical impedance sensor arrangement can be realized with a low outlay.

In the case of the vertical impedance arrangement of the invention, it is possible to form two surfaces or surface regions of the first and second electrically conductive structures that are essentially oriented parallel to one another, which surfaces are arranged at a predetermined distance from one another in the vertical direction of the vertical impedance arrangement.

The vertical impedance arrangement may be set up as a submicron vertical impedance arrangement, i.e., with at least one structural dimension of less than one micrometer (e.g. minimum distance between the first and second electrically conductive structures).

A minimum distance between the first and second electrically conductive structures may be found exclusively by means of the spacer. A minimum distance between the first and second electrically conductive structures may be defined by means of precisely one spacer. The spacer is preferably formed in one piece and/or from one material and/or from an electrically insulating material. The spacer may exclusively comprise a single material, preferably an electrically insulating material.

A minimum distance between the first and second uncovered surfaces is preferably at most 200 nm, further preferably at most 50 nm.

The capture molecules may be oligonucleotides, DNA half strands, peptides, proteins or low molecular weight compounds. The capture molecules may be organic or inorganic molecules.

A porous permeation layer may be arranged between at least one of the electrically conductive structures and the capture molecules and has pores of a predetermined size, in such a way that molecules whose size is less than or equal to the predetermined pore size can diffuse through the porous material, whereas molecules whose size exceeds the predetermined pore size cannot diffuse through the porous material.

Many biological molecules are very sensitive to free electrical charges or to extreme pH values. In a region directly surrounding the electrically charged sensor electrodes, that is to say the electrically conductive structures, very high or very low pH values and also free electrical charge carriers may occur, which may damage biological material. By means of a porous permeation layer that at least partially sheaths the sensor electrodes, macromolecular biomolecules having extents greater than the pore size of the permeation layer are protected from direct contact with the electrical charge carriers or from a milieu with an extreme pH value. By contrast, small ions or molecules (for example sodium chloride, water) can penetrate to the electrode surface.

Furthermore, the vertical impedance sensor arrangement of the invention may have at least one protective layer on at least one part of the first and/or of the second surface region, which protective layer is set up in such a way that the surface sections covered with the protective layer are free of a covering with capture molecules.

The capture molecules are often very expensive biological molecules that are difficult to obtain and are often present only in a small quantity. By virtue of a part of the uncovered surface sections of the electrically conductive structures being covered by means of a protective layer or by means of an encapsulation, specific surface regions on which capture molecules are immobilized can be prescribed in a targeted manner. The number of capture molecules required is thereby reduced.

The substrate is preferably a silicon substrate, a layer sequence comprising silicon and silicon nitride or a layer sequence comprising silicon and silicon oxide.

The first and/or the second electrically conductive structure may be produced from one or a combination of the materials gold, platinum, silver, silicon, aluminum and titanium.

Gold, in particular, is suitable as material for the electrically conductive structures for many applications since the gold-sulfur coupling is particularly advantageous chemically and since many capture molecules have sulfur-containing terminal groups, for example thiol groups (SH).

The spacer is preferably produced from an electrically insulating material. Preferably, the spacer is produced from silicon oxide (e.g. silicon dioxide) or silicon nitride. The spacer may be produced from one or a plurality of layers, each of which has one or a plurality of materials.

The protective layer may be produced from one or a combination of the materials silicon oxide and silicon nitride.

The first and/or the second electrically conductive structure may be formed as a conductor track, as a conductor plane, in a manner essentially running in meandering fashion or in a manner essentially running spirally.

The first and second electrically conductive structures may be arranged essentially parallel or perpendicular to one another.

The vertical impedance sensor arrangement of the invention may also have a plurality of first electrically conductive structures and/or a plurality of second electrically conductive structures.

These may be arranged in matrix form, for example, in order to form a sensor element in each crossover region. The sensor elements are preferably provided with different capture molecules that are sensitive to different particles to be detected.

Moreover, one of the electrically conductive structures may be provided as a conductor plane and the other electrically conductive structure may be provided as an arrangement of conductor tracks, which arrangement is preferably arranged parallel to the conductor plane.

Preferably, the vertical impedance sensor arrangement of the invention is set up as a biosensor for detecting macromolecular biomolecules.

The method according to the invention for producing a vertical impedance sensor arrangement is described below. Refinements of the vertical impedance sensor arrangement also apply to the method for producing a vertical impedance sensor arrangement.

The thickness of the spacer is preferably prescribed by means of a deposition method. Since a thickness of the spacer can be set very exactly by means of a deposition method and since the accuracy when setting the thickness of the spacer is particularly high with a deposition method, the structural dimensions that can be achieved are reduced according to the invention.

The spacer is preferably formed by means of an atomic layer deposition method (ALD method) or a chemical vapor phase epitaxy method (CVD method, “chemical vapor deposition”).

Exemplary embodiments of the invention are illustrated in the figures and are explained in more detail below.

### BRIEF DESCRIPTION OF THE DRAWINGS

FIGS. 1A and 1B show cross-sectional views of a sensor arrangement in accordance with the prior art in different operating states;

FIGS. 2A to 2C show cross-sectional views of another sensor arrangement in accordance with the prior art in different operating states;

FIG. 3A and 3B show further sensor arrangements in accordance with the prior art with different lateral dimensions of sensor electrodes;

FIGS. 4A to 4C show layer sequences at different points in time during a method according to the invention for producing a vertical impedance sensor arrangement in accordance with a first exemplary embodiment of the invention;

FIG. 5 shows a vertical impedance sensor arrangement in accordance with a second exemplary embodiment of the invention;

FIG. 6 shows a vertical impedance sensor arrangement in accordance with a third exemplary embodiment of the invention;

FIG. 7 shows a vertical impedance sensor arrangement in accordance with a fourth exemplary embodiment of the invention;

FIG. 8A shows a cross-sectional view along the section line I-I′—shown in FIG. 8B—of a vertical impedance sensor arrangement in accordance with a fifth exemplary embodiment of the invention;

FIG. 8B shows a perspective view of the vertical impedance sensor arrangement in accordance with the fifth exemplary embodiment of the invention as shown in FIG. 8A;

FIG. 9A shows a cross-sectional view along the section line II-II′—shown in FIG. 9B—of a vertical impedance sensor arrangement in accordance with a sixth exemplary embodiment of the invention; and

FIG. 9B shows a perspective view of the vertical impedance sensor arrangement in accordance with the sixth exemplary embodiment of the invention as shown in FIG. 9A.

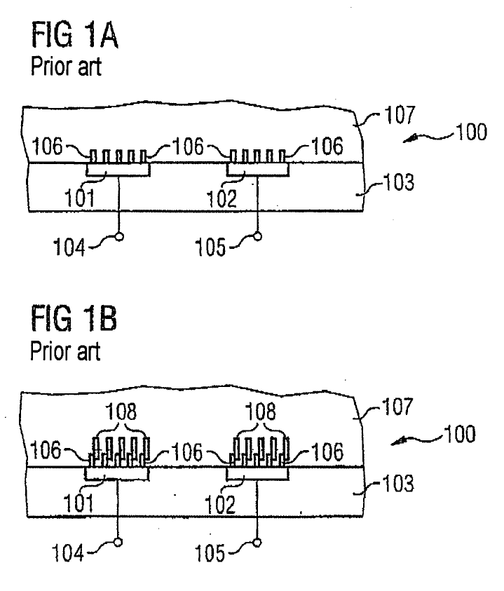

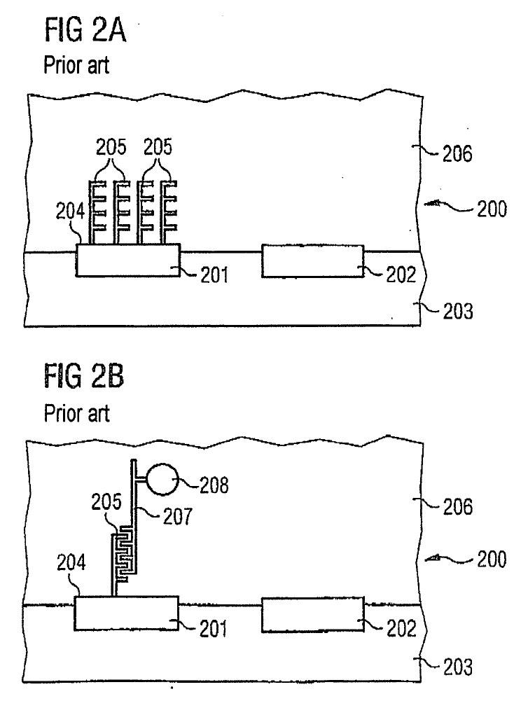

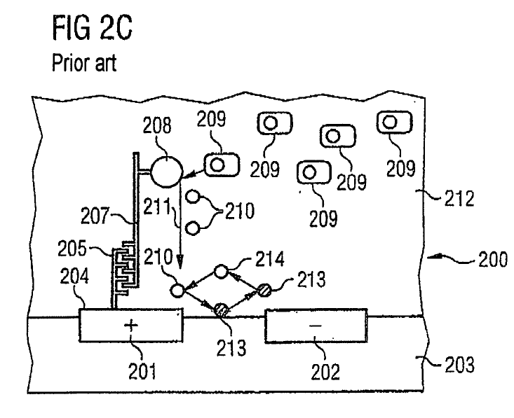

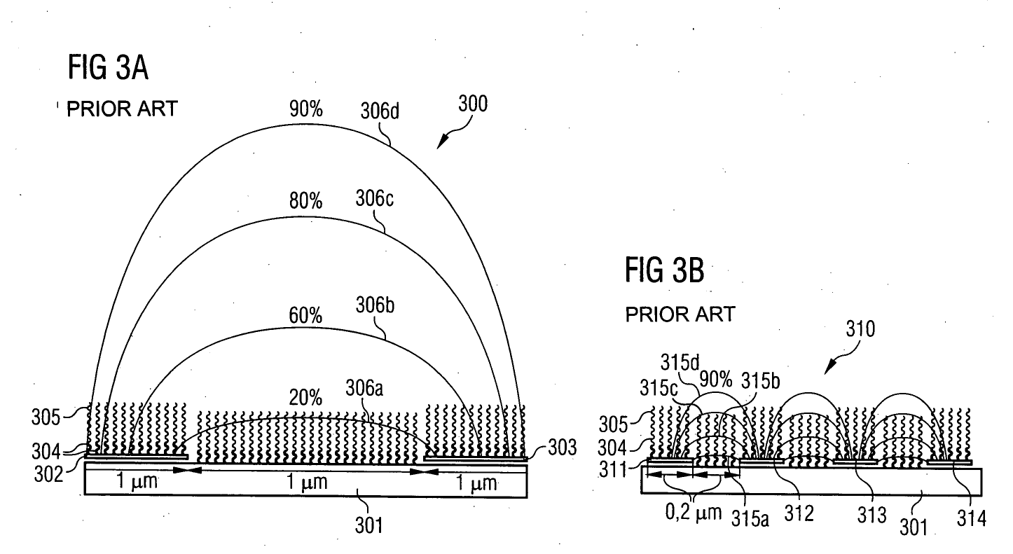

### DETAILED DESCRIPTION OF THE PREFERRED MODE OF THE INVENTION

It should be noted that in the description of the exemplary embodiments, those components of vertical impedance sensor arrangements which are included in different exemplary embodiments are provided with identical reference numerals.

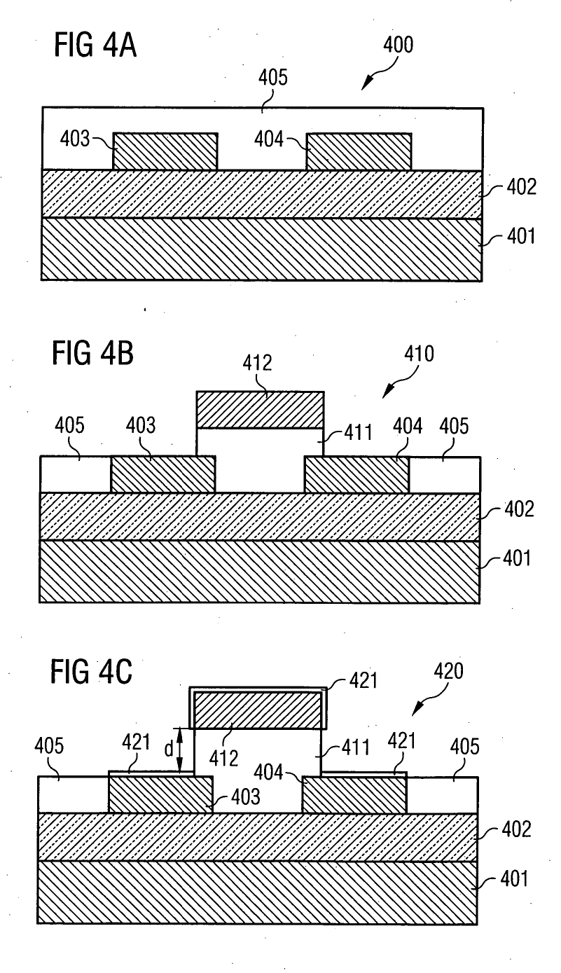

A description is given below, with reference to FIG. 4A to 4C, of an exemplary embodiment of the method according to the invention for producing a vertical impedance sensor arrangement.

In order to obtain the layer sequence 400 shown in FIG. 4A, a passivation layer 402 made of silicon nitride is deposited on a silicon wafer 401. Furthermore, a gold layer is deposited on the passivation layer 402 using a vapor deposition method and is patterned using a photolithography method (e.g. lift-off method). As a result, a first gold conductor track 403 and a second gold conductor track 404 remain on the passivation layer 402. A silicon dioxide layer 405 is subsequently deposited on the surface of the layer sequence thus obtained, using a CVD method (“chemical vapor deposition”). The surface of the silicon dioxide layer 405 is planarized using a CMP method (“chemical mechanical polishing”).

In order to obtain the layer sequence 410 shown in FIG. 4B, a further gold layer is deposited on the layer sequence 400. Using a photolithography method, the further gold layer is patterned (for example lift-off) and the silicon dioxide layer 405 is patterned by means of an RIE method (“reactive ion etching”) to leave the spacer 411 shown in FIG. 4B, by means of which a third gold conductor track 412 is spatially and electrically decoupled from the first and second gold conductor tracks 403, 404.

The layer sequence 420 shown in FIG. 4C is obtained by immobilizing DNA half strands 421 as capture molecules on uncovered surface regions of the first, second and third gold conductor tracks 403, 404, 412, said strands being set up in such a way that particles to be detected can hybridize with them.

A minimum distance d between the first gold conductor track 403 or the second gold conductor track 404, on the one hand, and the third gold conductor track 412, on the other hand, amounts to 50 nm. On account of this short distance, which is set highly precisely owing to the use of a CVD method as deposition method, the vertical impedance sensor arrangement 420 from FIG. 4C is a highly sensitive sensor for detecting biomolecules.

A description is given below, with reference to FIG. 5, of a vertical impedance sensor arrangement 500 in accordance with a second exemplary embodiment of the invention.

The vertical impedance sensor arrangement 500 differs from the vertical impedance sensor arrangement 420 essentially by the fact that before DNA half strands 421 are applied to uncovered surface regions of the gold conductor tracks 403, 404, 412, the spacer 411 and also the silicon dioxide regions 405 are etched back using a suitable etching method. The etching method is chosen in such a way that the etchant used has a high etching rate with respect to silicon dioxide material, whereas the etching rate with respect to gold material is very low. As a result, an etched-back spacer 501 and etched-back silicon dioxide regions 502 remain, whereas the gold regions 403, 404, 412 are protected against etching.

After this etching method has been carried out, uncovered surfaces of the gold conductor tracks 403, 404, 412 are provided with DNA half strands 421. On account of the etching-back, the active surface region, that is to say the surface region provided with capture molecules, of the conductor tracks 403, 404, 412 is increased, as a result of which the detection sensitivity is increased. In particular, by means of undercutting, it is possible to obtain mutually opposite surface regions—provided with DNA half strands 421—of the gold conductor tracks 403, 404, on the one hand, and of the gold conductor track 412, on the other hand, which surface regions are arranged, structurally close, parallel to one another.

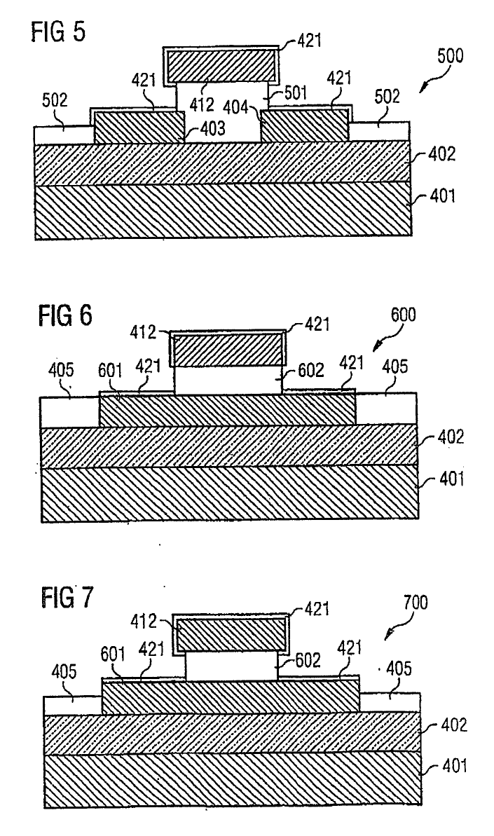

A description is given below, with reference to FIG. 6, of a vertical impedance sensor arrangement 600 in accordance with a third exemplary embodiment of the invention.

The vertical impedance sensor arrangement 600 differs from the vertical impedance sensor arrangement 420 essentially by the fact that after the first gold layer has been deposited, said gold layer is not patterned in such a way that a first and a second gold conductor track 403, 404 are formed thereby. Instead, the first gold layer is patterned in such a way that a single first gold conductor track 601 remains. The further method steps for forming the vertical impedance sensor arrangement 600 are then effected essentially analogously to the description relating to FIG. 4A to FIG. 4C.

In particular, a spacer 602 made of silicon dioxide is formed, which separates the gold conductor track 601 from the gold conductor track 412. Finally, DNA half strands 421 are immobilized on the surface of the layer sequence obtained.

A description is given below, with reference to FIG. 7, of a vertical impedance sensor arrangement 700 in accordance with a fourth exemplary embodiment of the invention.

The essential difference between the vertical impedance sensor arrangement 700 and the vertical impedance sensor arrangement 600 is that, in the case of the vertical impedance sensor arrangement 700, the components made of silicon dioxide 405, 602 are etched back before the immobilization of the DNA half strands 421 on uncovered surface regions of the gold conductor tracks 601, 412 using a suitable etching method. This increases the surface region of the first and third gold conductor tracks 601, 412 that is provided with capture molecules by comparison with the arrangement shown in FIG. 6, thereby achieving a higher area occupation with DNA half strands 421 and consequently a higher detection sensitivity. Mutually opposite, parallel surface regions—provided with DNA half strands 421—of the gold conductor tracks 601 and 412 are realized on account of the undercutting.

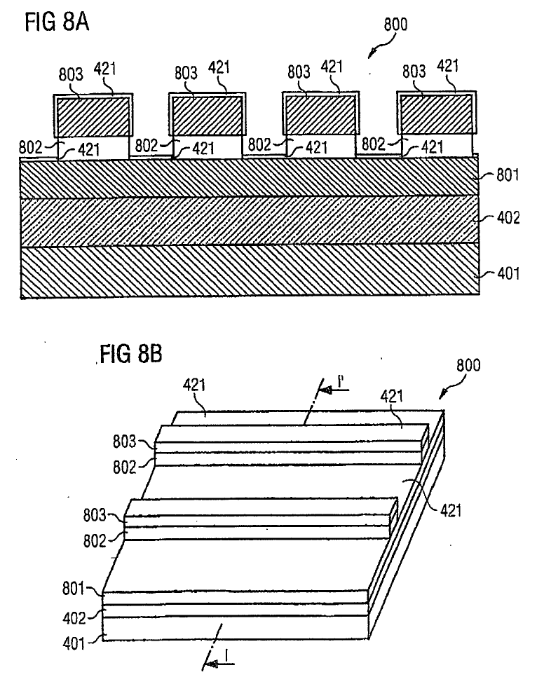

A description is given below, with reference to FIG. 8A, FIG. 8B, of a vertical impedance sensor arrangement 800 in accordance with a fifth exemplary embodiment of the invention.

FIG. 8B shows a perspective view of part of the vertical impedance sensor arrangement 800. The cross-sectional view of the vertical impedance sensor arrangement 800 as shown in FIG. 8A is taken along the section line I-I′.

A gold conductor plane 801 is formed on the silicon nitride passivation layer 402 that is in turn formed on the silicon substrate 401. First of all a silicon dioxide layer is deposited on said gold conductor plane and a second gold layer is deposited on said silicon dioxide layer. The last two layers are patterned jointly in such a way that the silicon dioxide tracks 802 and the gold conductor tracks 803 thereby remain. DNA half strands 421 are immobilized on the uncovered surfaces of the gold conductor plane 801 and the gold conductor tracks 802.

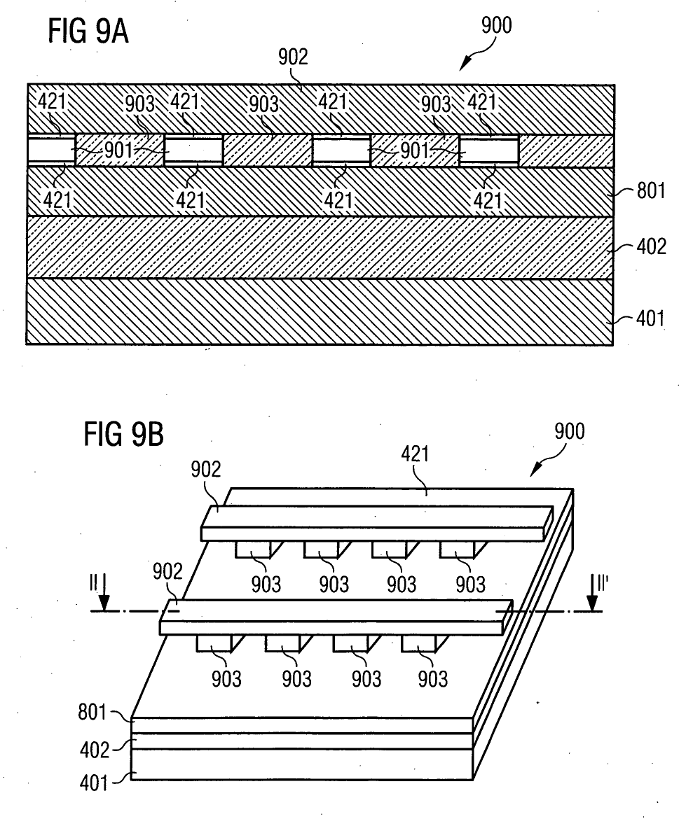

A description is given below, with reference to FIG. 9A, FIG. 9B, of a vertical impedance sensor arrangement 900 in accordance with a sixth exemplary embodiment of the invention.

FIG. 9B shows a perspective schematic view of part of the vertical impedance sensor arrangement 900, and FIG. 9A shows a cross-sectional view along the section line II-II′ shown in FIG. 9B.

In order to produce the vertical impedance sensor arrangement 900, a passivation layer 402 made of silicon nitride is deposited on the silicon wafer 401 and a gold conductor plane 801 is deposited on the passivation layer 402. A silicon dioxide layer is then deposited on the gold conductor plane 801 and patterned to form silicon dioxide tracks running perpendicular to the paper plane of FIG. 9A, so that silicon dioxide material is in the meantime included in particular in the voids 901 shown in FIG. 9A. A silicon nitride layer is deposited on this patterned layer sequence. A planar surface of the resulting layer sequence is produced using a CMP method. A further gold layer is deposited on said planar surface and patterned together with the underlying layer made of silicon nitride or made of silicon dioxide in such a way that the gold conductor tracks 902 remain in the manner shown in FIG. 9A, FIG. 9B. Clearly, the gold conductor tracks run essentially orthogonally with respect to the silicon dioxide tracks formed previously. Using a selective undercutting method, the silicon dioxide material is then removed from the voids 901 shown in figure.9A. The etchant is chosen in such a way that the etching rate is high for silicon dioxide material and very low for silicon nitride material, so that silicon nitride spacers 903 remain between the gold conductor plane 801 and the gold conductor tracks 902 in the manner shown in FIG. 9A, FIG. 9B.

In a further method step for forming the vertical impedance sensor arrangement 900, DNA half strands 421 are immobilized on uncovered gold surfaces.

1. A vertical impedance sensor arrangement, comprising:
a substrate;
a first electrically conductive structure having a first uncovered surface and being arranged in and/or on the substrate;
a spacer arranged above the substrate and/or at least partially on the first electrically conductive structure, said spacer being formed separately from the substrate, and the thickness of said spacer being defined by means of a deposition method;
a second electrically conductive structure having a second uncovered surface and being arranged on the spacer; and
capture molecules, which are immobilized on the first and on the second uncovered surface, are set up such that particles to be detected hybridize with said capture molecules.

2. The vertical impedance sensor arrangement as claimed in claim 1, wherein a minimum distance between the first and second uncovered surfaces is at most 200 nm.

3. The vertical impedance sensor arrangement as claimed in claim 1, wherein a minimum distance between the first and second uncovered surfaces is at most 50 nm.

4. The vertical impedance sensor arrangement as claimed in claim 1, wherein the capture molecules are oligonucleotides, DNA half strands, peptides, proteins, or low molecular weight compounds.

5. The vertical impedance sensor arrangement as claimed in claim 1, wherein a porous permeation layer is arranged between at least one of the electrically conductive structures and the capture molecules and has pores of a predetermined size, such that molecules whose size is less than or equal to the predetermined pore size diffuse through the porous material, whereas molecules whose size exceeds the predetermined pore size do not diffuse through the porous material.

6. The vertical impedance sensor arrangement as claimed in claim 1, further comprising at least one protective layer arranged on at least one part of the first and/or of the second uncovered surface, wherein the part or parts covered with the protective layer are free of capture molecules.

7. The vertical impedance sensor arrangement as claimed in claim 1, wherein the substrate is a silicon substrate, a layer sequence comprising silicon and silicon nitride, or a layer sequence comprising silicon and silicon oxide.

8. The vertical impedance sensor arrangement as claimed in claim 1, wherein the first and/or the second electrically conductive structures are/is produced from one or a combination of materials selected from the group consisting of gold, platinum, silver, silicon, aluminum, and titanium.

9. The vertical impedance sensor arrangement as claimed in claim 1, wherein the spacer is produced from one or a combination of materials selected from the group consisting of silicon oxide and silicon nitride.

10. The vertical impedance sensor arrangement as claimed in claim 6, wherein the protective layer is produced from one or a combination of materials selected from the group consisting of silicon oxide and silicon nitride.

11. The vertical impedance sensor arrangement as claimed in claim 1, wherein the first and/or the second electrically conductive structures are/is formed as a conductor track, as a conductor plane, in a manner essentially running in meandering fashion, or in a manner essentially running spirally.

12. The vertical impedance sensor arrangement as claimed in claim 1, wherein the first and second electrically conductive structures are arranged essentially parallel or perpendicular to one another.

13. The vertical impedance sensor arrangement as claimed in claim 1, further comprising a plurality of first electrically conductive structures and/or a plurality of second electrically conductive structures.

14. The vertical impedance sensor arrangement as claimed in claim 1, arranged as a biosensor for detecting macromolecular biomolecules.

15. A method for producing a vertical impedance sensor arrangement, comprising the steps of:
forming a first electrically conductive structure, which has a first uncovered surface, in and/or on a substrate;
forming a spacer above the substrate and/or at least partially on the first electrically conductive structure, said spacer being formed separately from the substrate, and the thickness of said spacer being defined by means of a deposition method;
forming a second electrically conductive structure, which has a second uncovered surface, on the spacer; and
immobilizing capture molecules on the first and second uncovered surfaces, said capture molecules being set up such that particles to be detected hybridize with said capture molecules.

16. The method as claimed in claim 15, wherein the spacer is formed by means of an atomic layer deposition method or a chemical vapor phase epitaxy method.
